(19) United States
(12) Patent Application Publication
(10) Pub. No.: US 2012/0101169 A1
(43) Pub. Date: Apr. 26, 2012

(54) METHODS OF PROVIDING ANTICOAGULATION EFFECTS IN SUBJECTS

(75) Inventor: Amale HAWI, Ridgefield, CT (US)

(73) Assignee: Penwest Pharmaceuticals Co., Chadds Ford, PA (US)

(21) Appl. No.: 13/180,185

(22) Filed: Jul. 11, 2011

Related U.S. Application Data
(60) Provisional application No. 61/364,325, filed on Jul. 14, 2010.

(51) Int. Cl. A61K 31/122; C07C 49/713; A61P 9/00; C07C 39/11; A61P 7/02; A61P 9/10; A61K 31/05; C07C 39/24

(52) U.S. Cl. 514/690; 514/734; 568/377; 568/765; 568/763

(57) ABSTRACT

The present invention is directed to methods of providing anticoagulation effects in subjects in need thereof, comprising administering to the subjects at least twice a day compounds of the present invention, stereoisomers, and racemates thereof.

### FIELD OF THE INVENTION

The present invention relates to methods of providing anticoagulation effects in subjects in need thereof, comprising administering to the subjects compounds of the present invention, stereoisomers, and racemates thereof.

### BACKGROUND OF THE INVENTION

Anticoagulation therapy is used to reduce or prevent the formation of blood clots in subjects who are experiencing, or are at risk of experiencing, blood clots which can result in a complete or partial obstruction of the flow of blood in a subject, including subjects having stroke, myocardial infarction, complications associated with cardiac valve replacement, and combinations thereof.

Anticoagulation therapy has been previously accomplished by administration of warfarin sodium to a subject either orally or by injection. See physician's label for COUMADIN® (Bristol-Myers Squibb Co., Princeton, N.J., revised January 2010). Warfarin sodium is an antagonist of vitamin K, a necessary element in the synthesis of blood clotting factors II, VII, IX and X, as well as naturally occurring endogenous anticoagulant proteins C and S.

Heparin sodium is another anticoagulation therapy that is administered via injection. See physician's label for heparin sodium (APP Pharmaceuticals, LLC, Schaumburg, Israel). Heparin sodium exerts its anticoagulant action by accelerating the activity of antithrombin III (ATIII).

Vitamin E quinone has also been known to exhibit anticoagulation properties. See Dowd, P., et al., Proc. Natl. Acad. Sci. 92: 8171-8175 (1995). It is speculated that vitamin E quinone acts to directly inhibit the vitamin K-dependent carboxylase that controls blood clotting.

Warfarin sodium and heparin sodium therapies can require substantial dosage maintenance through periodic determinations of blood clotting times in a subject. For example, administration of heparin sodium requires determination of blood clotting times every four hours in the early stages of treatment. See physician's label for heparin sodium (APP Pharmaceuticals, LLC, Schaumburg, Israel). This is due, in part, because the coagulation status of subjects receiving heparin sodium treatment is in constant flux. Valenstein, P., et al., Archives of Pathology and Laboratory Medicine, 128: 4, 397-402 (2003). Additionally, the physician's label for COUMADIN® (wafarin sodium) discloses significant bleeding risks and indicates that periodic determination of blood clotting times in a subject is essential. See physician's label for COUMADIN® (Bristol-Myers Squibb Co., Princeton, N.J., revised January 2010).

Thus, there exists a need for anticoagulation therapy which provides predictable clinical results and minimizes dosage maintenance in subjects.

### BRIEF SUMMARY OF THE INVENTION

The present invention is directed to a method of providing an anticoagulation effect in a subject in need thereof, comprising administering to the subject at least twice a day a compound of formula (I):

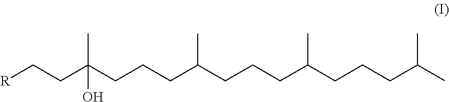

wherein R is selected from:

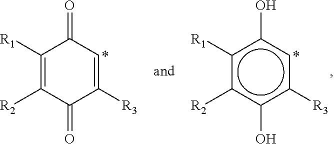

wherein the * indicates the point of attachment of R; and R₁, R₂, and R₃ are independently selected from H, C₁-C₆ alkyl, and C₁-C₆ haloalkyl. In some embodiments, if any one of R₁, R₂, and R₃ is H, then at least one other of R₁, R₂, and R₃ is neither H nor methyl.

In some embodiments, the compound is administered at least three times a day.

In some embodiments, a total daily dosage of 0.2 g to 12 g of the compound is administered to the subject. In some embodiments, 0.1 g to 6 g of the compound is administered to the subject at least twice a day. In some embodiments, 0.5 g to 4 g of the compound is administered to the subject at least twice a day. In some embodiments, 0.1 g to 4 g of the compound is administered to the subject three times a day. In some embodiments, 0.3 g to 2 g of the compound is administered to the subject three times a day.

In some embodiments, R is:

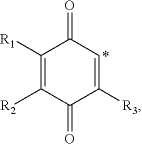

and R₁, R₂, and R₃ are independently selected from H or C₁-C₂ alkyl.

In some embodiments, the compound of formula (I) is:

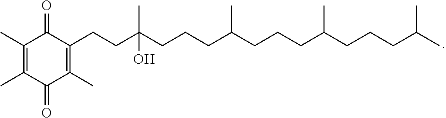

In some embodiments, the compound of formula (I) is:

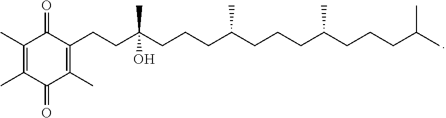

The present invention is also directed to a method of treating thrombosis in a subject in need thereof, comprising administering to the subject at least twice a day a compound of formula (I):

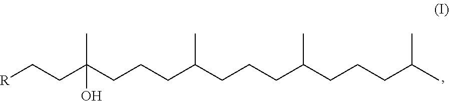

wherein R is selected from:

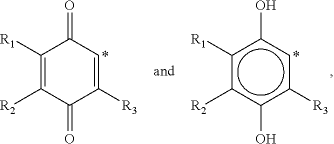

wherein the * indicates the point of attachment of R; and R₁, R₂, and R₃ are independently selected from H, C₁-C₆ alkyl, and C₁-C₆ haloalkyl. In some embodiments, if any one of R₁, R₂, and R₃ is H, then at least one other of R₁, R₂, and R₃ is neither H nor methyl.

In some embodiments, the thrombosis is selected from the group consisting of venous thrombosis, deep vein thrombosis, renal vein thrombosis, arterial thrombosis, and combinations thereof.

The present invention is also directed to a method of treating thrombosis in a subject in need thereof, comprising administering to the subject at least twice a day 0.1 g to 6 g of a compound of formula (I), wherein the compound is:

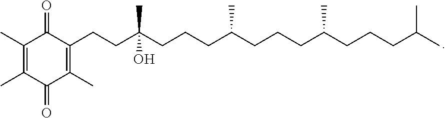

The present invention is also directed to a method of treating a condition selected from the group consisting of stroke, myocardial infarction, complications associated with cardiac valve replacement, and combinations thereof in a subject in need thereof, the method comprising administering to the subject at least twice a day a compound of formula (I):

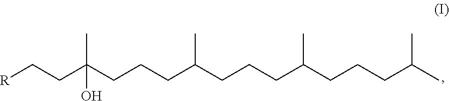

wherein R is selected from:

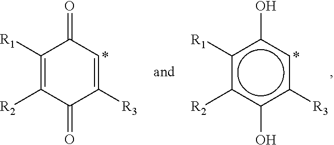

wherein the * indicates the point of attachment of R; and R₁, R₂, and R₃ are independently selected from H, C₁-C₆ alkyl, and C₁-C₆ haloalkyl. In some embodiments, if any one of R₁, R₂, and R₃ is H, then at least one other of R₁, R₂, and R₃ is neither H nor methyl.

In some embodiments, the compound of formula (I) is administered to the subject orally, nasally, via inhalation, parenterally, subcutaneously, intramuscularly, transdermally, or buccally. In some embodiments, an oral dosage form comprising a compound of formula (I) is administered to the subject.

In some embodiments, the method of the present invention further comprises, measuring in a subject at least one coagulation factor selected from the group consisting of Factor I, Factor II, Factor V, Factor VII, Factor X, Protein C, Protein S, antithrombin, platelet function, and combinations thereof. In some embodiments, the method of the present invention further comprises measuring the international normalized ratio (INR), prothrombin time (PT), activated partial thromboplastin time (aPTT), and combinations thereof in the subject.

In some embodiments, the compound of formula (I) is a stereoisomer thereof. In some embodiments, the compound of formula (I) is a racemate thereof.

In some embodiments, the method comprises administering a compound of formula (I) to a subject for a period of less than 20 days.

The present invention is also directed to a therapeutic package comprising (a) greater than seven dosage forms, each dosage form comprising 0.1 g to 6 g of a compound of formula (I):

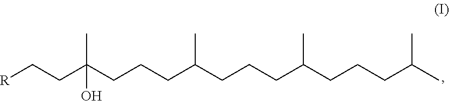

wherein R is selected from:

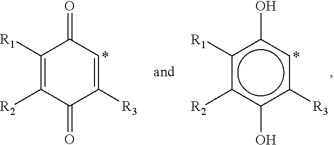

wherein the * indicates the point of attachment of R; and R₁, R₂, and R₃ are independently selected from H, C₁-C₆ alkyl, and C₁-C₆ haloalkyl, and (b) a label comprising directions for administering the compound to a subject according to the methods of the present invention. In some embodiments, if any one of R₁, R₂, and R₃ is H, then at least one other of R₁, R₂, and R₃ is neither H nor methyl.

### BRIEF DESCRIPTION OF THE DRAWINGS

FIG. 1 is a graphical description of the average measured INR values in subjects administered the compounds of the present invention. The solid lines represent INR values of subjects administered 0.67 g of (R,R,R) α-tocopherol quinone three times a day for a period of 144 hours. The dashed lines represent INR values or subjects administered 0.33 g of (R,R,R) α-tocopherol quinone three times a day for a period of 144 hours. INR values of each subject were measured at 24 hour intervals during the course of administration. Grade 2 refers to an INR value in a subject of 1.65 to 2.2. Grade 3 refers to an INR value in a subject greater than 2.2. Solid line 101 represents the subject with the highest measured INR value. Solid line 102 represents the subject with the lowest measured INR value. Dashed line 103 represents the subject with the highest measured INR value. Dashed line 104 represents the subject with the lowest measured INR value.

FIG. 2 is a graphical description of the INR values measured in subjects administered 0.75 g of (R,R,R) α-tocopherol quinone twice a day for a period of 336 hours. INR values of each subject were measured at 24 hour intervals during the course of administration.

FIG. 3 is a graphical description of the INR values measured in subjects administered (R,R,R) α-tocopherol quinone. The solid lines represent INR values in subjects administered 0.75 g of (R,R,R) α-tocopherol quinone twice a day for a period of 336 hours. The dashed lines represent INR values in subjects administered 0.5 g of (R,R,R) α-tocopherol quinone twice a day for a period of 336 hours. INR values of each subject were measured at 24 hour intervals during the course of administration.

FIG. 4 is a graphical description of the INR values measured in subjects administered (R,R,R) α-tocopherol quinone. The solid lines represent INR values in subjects administered 0.5 g of (R,R,R) α-tocopherol quinone twice a day for a period of 168 hours. The dashed lines represent INR values in subjects administered 0.33 g of (R,R,R) α-tocopherol quinone three times a day for a period of 168 hours. INR values of each subject were measured at 24 hour intervals during the course of administration.

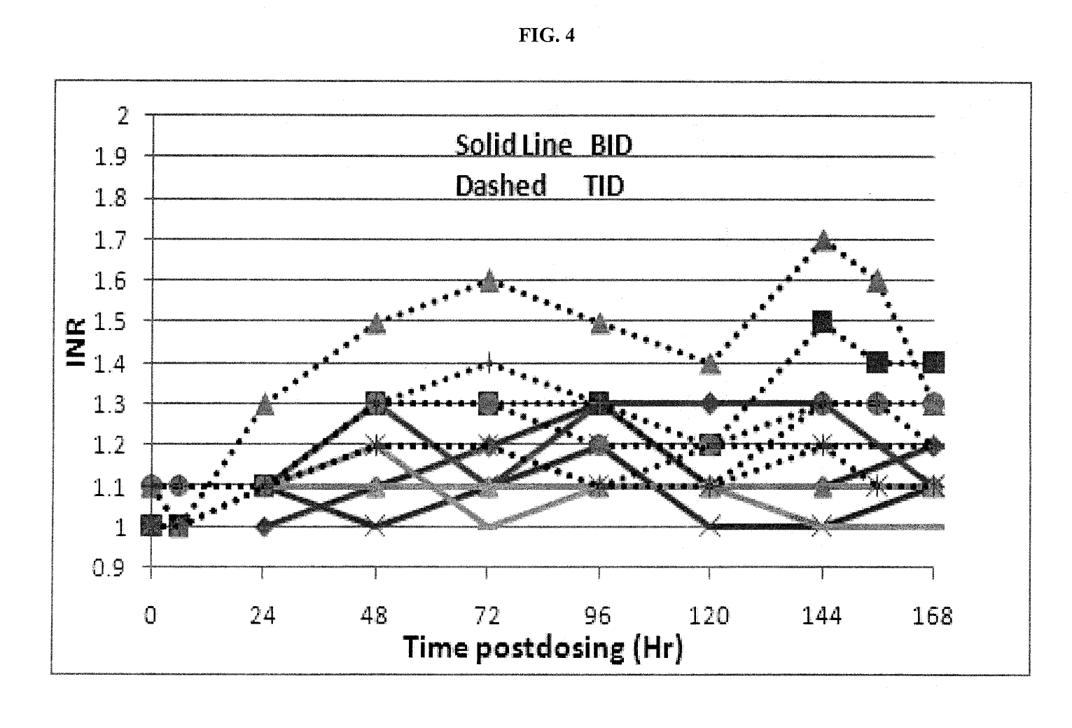

### DETAILED DESCRIPTION OF THE INVENTION

The present invention is directed to a method of providing an anticoagulation effect in a subject in need thereof, comprising administering to the subject at least twice a day a compound of formula (I).

The present invention is also directed to a method of treating thrombosis in a subject in need thereof, comprising administering to the subject at least twice a day a compound of formula (I).

The present invention is also directed to a method of treating a condition selected from the group consisting of stroke, myocardial infarction, complications associated with cardiac valve replacement, and combinations thereof in a subject in need thereof, the method comprising administering to subject at least twice a day a compound of formula (I).

A compound of formula (I) is:

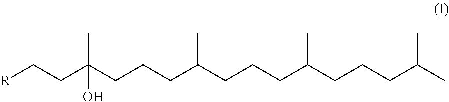

wherein R is selected from:

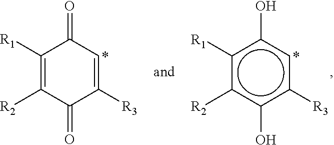

wherein the * indicates the point of attachment of R; and R₁, R₂, and R₃ are independently selected from H, C₁-C₆ alkyl, and C₁-C₆ haloalkyl. In some embodiments, R is

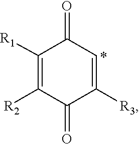

and R₁, R₂, and R₃ are independently selected from H and C₁-C₂ alkyl. In some embodiments, if any one of R₁, R₂, and R₃ is H, then at least one other of R₁, R₂, and R₃ is neither H nor methyl.

In some embodiments, the compound of formula (I) is:

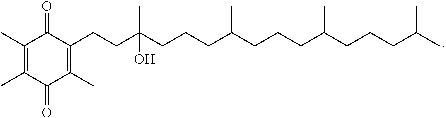

In some embodiments, the compound of formula (I) is:

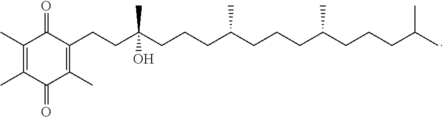

In some embodiments, the compound of formula (I) is a stereoisomer thereof. In some embodiments, the compound of formula (I) is a racemate thereof.

The present invention is directed to methods of providing an anticoagulation effect in a subject in need thereof, comprising administering to the subject at least twice a day a compound of formula (I) as described above. As used herein, the term “providing an anticoagulation effect” refers to preventing, inhibiting, or prolonging blood coagulation in a subject. “Blood coagulation” refers to the process by which blood forms clots in a subject. “Blood clots,” “blood clotting,” or “thrombus” refers to the aggregation of blood cells and/or platelets in the circulatory system of a subject. In some embodiments, a blood clot can partially or completely block the flow of blood in a subject. “Circulatory system” refers to the organ system in the subject comprising the heart, blood vessels, arteries, veins, capillaries, and blood. Thus, in some embodiments, the present invention is directed to methods of preventing, inhibiting, or prolonging the formation of blood clots in a subject which result in a partial or complete obstruction of the flow of blood in the circulatory system of the subject.

As used herein, a “subject” refers to a human or non-human animal, to which a the compound of formula (I) is administered. In some embodiments, the subject is a domesticated animal, a herd animal, or an animal in captivity, e.g., present in a zoo. In some embodiments, the subject is a female human. In some embodiments, the subject is a male human.

In some embodiments, the subject is a “subject in need thereof.” A subject in need thereof refers to an individual for whom it is desirable to treat, i.e., a subject who has experienced, or is experiencing, blood clots which can result in a partial or total obstruction of the flow of blood in the subject. Subjects in need thereof can also include subjects who are in need of treatment of prophylaxis of blood clotting as determined by one of skill in the art. In some embodiments, subjects in need thereof include subjects who have experienced, or are experiencing, stroke, myocardial infarction, cardiac valve replacement surgery, or combinations thereof. In some embodiments, subjects in need thereof include subjects who are preparing to undergo surgery, or subjects who have just underwent surgery, wherein an anti-coagulation or anti-thrombotic effect is desired.

As used herein, “administering” or “administration” refers to the process of introducing a compound of formula (I) to a subject. In some embodiments, administering means releasing an amount of a compound of formula (I) from a dosage form to a subject. Various modes of administration can be used in the present invention. For example, a compound of formula (I) can be administered to a subject orally, nasally, via inhalation, parenterally, transdermally, or buccally. In some embodiments, an oral dosage form comprising a compound of formula (I) is administered to the subject.

“Orally” refers to administration of a compound of formula (I) through the gastrointestinal tract. Non-limiting examples of suitable oral dosage forms for use with the methods of the present invention include tablets, capsules, elixirs, syrups, cachets, pellets, pills, powders and granules. In some embodiments, the oral dosage form is a capsule, elixir, or syrup. In some embodiments, the composition comprising a compound of formula (I) is inside a gel capsule.

“Nasally” refers to the administration of a compound of formula (I) through the nasal mucous membrane to the bloodstream for systemic delivery. “Inhalation” refers to the administration of a compound of formula (I) through the lungs to the bloodstream for systemic delivery. Non-limiting examples of suitable nasal or inhalation dosage forms for use with the methods of the present invention include inhalers, insufflators, and aerosol sprays. Aerosol spray presentation can be achieved from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. In some embodiments, the compound of formula (I) is nasally administered to the subject in liquid form, e.g., via a nasal mist or spray.

“Parenterally” refers to administration of a compound of formula (I) to a subject through means other than through the gastrointestinal tract or the lungs. Non-limiting examples of suitable parenteral dosage forms for use with the methods of the present invention include intravenous, intramuscular, and subcutaneous formulations. “Intravenous” refers to administration of a compound of formula (I) to a subject through the veins of the subject. “Subcutaneous” refers to administration of a compound of formula (I) to a subject through tissues or blood vessels immediately below the skin. “Intramuscularly” refers to administration of a compound of formula (I) to a subject through direct absorption by muscle tissues surrounding a subcutaneous dosage form without passing through a mucosal or dermal membrane. Non-limiting examples of suitable intravenous, subcutaneous, or intramuscular dosage forms for use with the methods of the present invention include intravenous formulations (e.g., oil-in-water emulsions or water-in-oil emulsions) and implantable dosage forms.

“Transdermally” refers to administration of a compound of formula (I) across a dermal membrane. “Buccally” refers to administration of a compound of formula (I) across the mucosa or tissue of the mouth. In some embodiments of the present invention, a compound of formula (I) is administered via a transdermal or buccal dosage form. The transdermal or buccal dosage form can be occlusive or non-occlusive. Non-limiting examples of suitable transdermal or buccal dosage forms for use with the present invention include a patch, an adhesive patch, a reservoir dosage form, a matrix dosage form, a multi-laminar patch, a non-occlusive patch, a bioadhesive tablet, and a bioadhesive plaster. Transdermal and buccal dosage forms for use with the methods of the present invention can further comprise a bio-adhesive layer useful to adhere the dosage form to the dermis or mucosa of a subject.

In some embodiments, a dosage form containing the compound of formula (I) further comprises an excipient. As used herein, an “excipient” refers to a substance that is used in the formulation of the intravaginal device of the present invention, and, by itself, generally has little or no therapeutic value. One of skill in the art will recognize that a wide variety of pharmaceutically acceptable excipients can be used, including those listed in the Handbook of Pharmaceutical Excipients, Pharmaceutical Press 4th Ed. (2003) and Remington: The Science and Practice of Pharmacy, Lippincott Williams & Wilkins, 21st Ed. (2005). As used herein, the term “pharmaceutically acceptable” refers to those compounds, materials, and/or compositions which are, within the scope of sound medical judgment, suitable for contact with the tissues of human beings and animals without excessive toxicity, irritation, allergic response, or other possible complications commensurate with a reasonable benefit/risk ratio. In some embodiments, the compounds of formula (I) are liquid at room temperature. Thus, in some embodiments, the excipients of the present invention are used to add viscosity or solidify a composition comprising the compound of formula (I). Examples of excipients can include, e.g., polyethylene glycol glycerides composed of mono-, di-, and triglycerides, and mono- and diesters of polyethylene glycol (Gelucire®, Gattefossé, Canada, Montreal, Canada). Excipients can also include anti-oxidants. Antioxidants refer to synthetic or natural compounds which prevent or reduce the oxidation of a compound of formula (I). Non-limiting examples of antioxidants include BHT, BHA, gallic acid, propyl gallate, ascorbic acid, and ascorbyl palmitate. Excipients can also include flavorants including natural and synthetic sweeteners, flavor oils (i.e., peppermint oil, spearmint oil, cinnamon oil, citrus oil, etc.), and combinations thereof.

Various amounts of the compound of formula (I) can be administered to a subject. The amount of compound to be administered to a subject can be determined by the nature of the symptom and/or the characteristics of the subject, e.g., weight, age, health, etc. In some embodiments, the amount of compound administered to a subject can be determined by a person of skill in the art. One of skill in the art can perform pharmacokinetic studies and use the results thereof to adjust the dosage amount to a suitable level, or determine an appropriate dosage amount based on systematically varying the dosage amount administered to a subject and monitoring the coagulation effect on the subject after the administration. Appropriate animal studies can be performed to determine an appropriate dosage amount. As used herein, “one of skill in the art” includes, for example, a physician, a physician's assistant, a nurse practitioner, a pharmacist, pharmacologist, pharmacokineticist and a customer service representative.

In some embodiments, a total daily dosage of 0.2 g to 12 g of a compound of formula (I) is administered to a subject. As used herein, the terms “total daily dosage,” “daily dosage level,” “daily dosage amount,” and “daily dose” refer to the total amount of a compound of formula (I) administered to a subject per day, i.e., per 24 hour period. Thus, for example, administration to a subject at a “total daily dosage” of 1 g of a compound of formula (I) means that a subject receives a total of 1 g of a compound of formula (I) on a daily basis, whether the compound of formula (I) is administered as a single 1 g dose or, e.g., two separate 0.5 g doses. In some embodiments, a total daily dosage of 0.3 g to 10 g of a compound of formula (I) is administered to a subject. In some embodiments, a total daily dosage of 0.4 g to 8 g of a compound of formula (I) is administered to a subject. In some embodiments, a total daily dosage of 0.5 g to 7 g of a compound of formula (I) is administered to a subject. In some embodiments, a total daily dosage of 0.6 g to 6 g of a compound of formula (I) is administered to a subject. In some embodiments, a total daily dosage of 0.7 g to 5 g of a compound of formula (I) is administered to a subject. In some embodiments, a total daily dosage of 0.8 g to 4 g of a compound of formula (I) is administered to a subject. In some embodiments, a total daily dosage of 0.9 g to 3 g of a compound of formula (I) is administered to a subject. In some embodiments, a total daily dosage of 1 g to 2 g of a compound of formula (I) is administered to a subject.

In some embodiments, the total daily dosage of a compound of formula (I) can be administered to a subject in multiple doses. In some embodiments, each dose in the multiple doses has the same dosage amount of a compound of formula (I) as the other doses in the multiple doses, e.g., if the total daily dosage administered to a subject is 1.5 g, administered as three distinct doses, each distinct dose has 0.5 g of a compound of formula (I). In some embodiments, each dose in the multiple doses has different dosage amounts of a compound of formula (I) as the other doses in the multiple doses, e.g., if the total daily dosage administered to a subject is 2 g, administered as three distinct doses, one distinct dose 0.4 g, a second distinct dose is 0.6 g, and a third distinct dosage is 1 g of a compound of formula (I).

In some embodiments, a total daily dosage of 6 g of a compound of formula (I) is administered to a subject. In some embodiments, a total daily dosage of 5 g of a compound of formula (I) is administered to a subject. In some embodiments, a total daily dosage of 4 g of a compound of formula (I) is administered to a subject. In some embodiments, a total daily dosage of 3 g of a compound of formula (I) is administered to a subject. In some embodiments, a total daily dosage of 2 g of a compound of formula (I) is administered to a subject. In some embodiments, a total daily dosage of 1.5 g of a compound of formula (I) is administered to a subject.

In some embodiments, a compound of formula (I) is administered to a subject at least once daily. In some embodiments, a compound of formula (I) is administered to a subject at least BID. As used herein, “BID” refers to Bis in Die, twice a day, or two times a day. Thus, when a compound of formula (I) is administered to a subject at least BID, the compound of formula (I) is administered to the subject at least twice a day. In some embodiments, the compound of formula (I) is administered to the subject approximately every 10 to 14 hours, e.g., every 12 hours.

In some embodiments, 0.1 g to 6 g of a compound of formula (I) is administered to the subject at least twice a day. In some embodiments, 0.2 g to 5 g of a compound of formula (I) is administered to the subject at least twice a day. In some embodiments, 0.5 g to 4 g of a compound of formula (I) is administered to the subject at least twice a day. In some embodiments, 0.6 g to 3 g of a compound of formula (I) is administered to the subject at least twice a day. In some embodiments, 0.7 g to 2 g of a compound of formula (I) is administered to the subject at least twice a day.

In some embodiments, a compound of formula (I) is administered to a subject at least TID. As used herein, “TID” refers to Ter in Die, thrice a day, or three times a day. Thus, when a compound of formula (I) is administered to a subject at least TID, the compound of formula (I) is administered to the subject at least three times a day. In some embodiments, the compound of formula (I) is administered to the subject approximately every 6 to 10 hours, e.g., every 8 hours.

In some embodiments, 0.1 g to 4 g of a compound of formula (I) is administered to the subject three times a day. In some embodiments, 0.2 g to 3 g of a compound of formula (I) is administered to the subject three times a day. In some embodiments, 0.3 g to 2 g of a compound of formula (I) is administered to the subject three times a day. In some embodiments, 0.4 g to 1 g of a compound of formula (I) is administered to the subject three times a day. In some embodiments, 0.5 g to 1.5 g of a compound of formula (I) is administered to the subject three times a day.

In some embodiments, a compound of formula (I) is administered to the subject at least four times a day. In some embodiments, the compound of formula (I) is administered to the subject approximately every 4 to 8 hours, e.g., every 6 hours. In some embodiments, a compound of formula (I) is administered to the subject at least five times a day. In some embodiments, a compound of formula (I) is administered to the subject concurrently with meals. In some embodiments, a compound of formula (I) is administered to the subject once in the morning and once at night.

In some embodiments, the duration of the administration depends on the subject's condition, disorder, or disease. In some embodiments, a compound of formula (I) is administered continuously, i.e., the total daily dosage of the compound is administered on consecutive days without interruption between days.

A compound of formula (I) can be administered to a subject for various periods of time. In some embodiments, a compound of formula (I) is administered to a subject for at least 3 days. In some embodiments, the compound of formula (I) is administered to a subject for at least 7 days. In some embodiments, the compound of formula (I) is administered to a subject for at least 14 days. In some embodiments, the compound of formula (I) is administered to a subject for at least 1 month. In some embodiments, the compound of formula (I) is administered to a subject for at least 3 months. In some embodiments, the compound of formula (I) is administered to a subject for at least 6 months. In some embodiments, the compound of formula (I) is administered to a subject for at least 1 year. In some embodiments, a compound of formula (I) is administered to a subject for the remainder of the subject's life.

In some embodiments, a compound of formula (I) is administered to a subject for less than 12 months. In some embodiments, a compound of formula (I) is administered to a subject for less than 10 months. In some embodiments, a compound of formula (I) is administered to a subject for less than 6 months. In some embodiments, a compound of formula (I) is administered to a subject for less than 3 months. In some embodiments, a compound of formula (I) is administered to a subject for less than 1 month. In some embodiments, a compound of formula (I) is administered to a subject for less than 20 days. In some embodiments, a compound of formula (I) is administered to a subject for less than 14 days.

In some embodiments, a compound of formula (I) is administered to a subject in a regimen comprising administering the compound to the subject for a first period of time, followed by a break in administration for a second period of time, and then these two steps are repeated at least once. For example, in some embodiments, a compound of formula (I) is administered to a subject for at least 14 days, followed by a break in administration for a period of at least 7 days, followed by administration for a period of at least 14 days, followed by a break in administration for a period of at least 7 days. As used herein, the term “break in administration” refers to a period of time in which a compound of formula (I) is not administered to a subject.

The present invention is also directed to methods of treating thrombosis in a subject in need thereof, comprising administering to the subject at least twice a day a compound of formula (I) as described above. As used herein, the term “thrombosis” refers to the coagulation of the blood in the circulatory system of the subject which results in a partial or complete obstruction of the flow of blood in the circulatory system of the subject. In some embodiments, the thrombosis is selected from the group consisting of venous thrombosis, deep vein thrombosis, renal vein thrombosis, arterial thrombosis, thromboembolism, and combinations thereof. “Venous thrombosis” refers to the formation of a blood clot within a vein of the subject. “Deep vein thrombosis” refers to the formation of blood clot within the leg and arm veins of the subject, such as the femoral vein or the popliteal vein. “Renal vein thrombosis” refers to the formation of a blood clot within a renal vein of the subject. “Arterial thrombosis” refers to the formation of a blood clot within an artery of the subject. “Thromboembolism” refers to the migration of a blood clot from one part of the circulatory system of a subject which can result in a partial or complete obstruction of the flow of blood in another part of the circulatory system in the subject.

As used herein the terms “treat,” “treatment,” and “treating” refer to both therapeutic treatment and prophylactic or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms; diminishment of the extent of a condition, disorder or disease; stabilization (i.e., not worsening) of the state of a condition, disorder or disease; delay in the onset or slowing of a condition, disorder or disease progression; amelioration of a condition, disorder or disease state; remission (whether partial or total), whether detectable or undetectable; and enhancement or improvement of a condition, disorder or disease. Treatment includes eliciting a clinically significant response, without excessive levels of side effects. Treatment also includes prolonging survival as compared to expected survival if not receiving treatment.

The methods of the present invention are also directed to treating a subject at risk of a condition selected from the group consisting of stroke, myocardial infarction, complications associated with cardiac valve replacement, and combinations thereof, the methods comprising administering to a subject a compound of formula (I) as described above. In some embodiments, a “subject at risk” refers to a subject with one or more risk factors for developing a disease, disorder, or condition. Non-limiting examples of risk factors include gender, age, weight, genetic predisposition, medical history, and lifestyle. In some embodiments, the existence of a risk factor can be determined by one of skill in the art.

“Stroke” refers to any condition arising from a disruption, decrease, or stoppage of blood or oxygen flow to any part of the brain. Non-limiting examples of stroke can include embolic stroke and thrombolic stroke. In some embodiments, a subject at risk of stroke is a subject includes subjects who have recently suffered from a stroke, have a family history of stroke, or are diagnosed to be at risk of stroke by one of skill in the art.

“Myocardial infarction” refers to damage to the heart resulting from a disruption, decrease, or stoppage of blood flow to the heart. In some embodiments, a subject at risk of myocardial infarction includes subjects who have recently suffered from a myocardial infarction or have been diagnosed to be at risk by one of skill in the art.

“Complications associated with cardiac valve replacement” refers to complications that can occur in a subject that has underwent, or will undergo, surgery to replace at least one cardiac valve. Non-limiting examples of complications associated with cardiac valve replacement can include blood clots, thromboembolism, infection, embolism, and combinations thereof.

In some embodiments, the methods of providing anticoagulation effect, treating thrombosis, and treating a subject at risk of a condition selected from the group consisting of stroke, myocardial infarction, complications associated with cardiac valve replacement, and combinations thereof, as described above, further comprise measuring in the subject at least one coagulation factor selected from the group consisting of Factor I, Factor II, Factor V, Factor VII, Factor IX, Factor X, Protein C, Protein S, antithrombin, platelet function, and combinations thereof. Coagulation factors and their functions are known in the art.

In some embodiments, the method of the present invention can provide an anticoagulation effect, or anti-thrombotic effect, in an individual for a short period of time, e.g., less than one month, less than three weeks, or less than two weeks. For example, in some embodiments, anticoagulation effect or anti-thrombotic effect can be provided to individual who has underwent a surgical procedure (e.g., hip surgery, knee surgery, etc.), by administration of the compound of formula (I) as described herein, e.g., at least twice daily for less than one month, less than three weeks, or less than two weeks.

Measuring any one coagulation factor in a subject can be achieved through a number of different blood coagulation assays. Blood coagulation assays and methods of using these assays are known in the art. Non-limiting examples of blood coagulation assays include activated partial thromboplastin time (aPTT), prothrombin time (PT), prothrombin ratio (PR), international normalized ratio (INR), fibrinogen testing, platelet count, and platelet function testing, e.g., PFA-100. In some embodiments, any one coagulation factor is measured before and after the initial administration of a compound of formula (I). In some embodiments, any one coagulation factor is measured in a subject at 1 hour, 2 hour, 3 hour, 6 hour, 12 hour, 24 hour, or 48 hour intervals after administration of the compound of formula (I) to the subject.

In some embodiments, the methods of providing anticoagulation effect, treating thrombosis, and treating a subject at risk of a condition selected from the group consisting of stroke, myocardial infarction, complications associated with cardiac valve replacement, and combinations thereof, as described above, further comprise measuring the international normalized ratio (INR), prothrombin time (PT), activated partial thromboplastin time (aPTT), or a combination thereof in the subject. The INR is the ratio of a subject's prothombin time (PTₜₑₛₜ) to the prothombin time of a normal (control) sample (PTₙₒᵣₘₐₗ), raised to the power of the International Sensitivity Index (ISI) value for the analytical system:

$$INR = {( \frac{{PT}_{test}}{{PT}_{normal}} )}^{ISI}$$

“Prothrombin time” or “PT” refers to the time it takes the blood plasma of a subject to clot after the addition of a thromboplastin reagent. “Thromboplastin reagent” refers to a standardized commercial product which is used in blood coagulation assays to measure blood clotting time. Non-limiting examples of thromboplastin reagents include RecombiPlasTin (Beckman Coulter, Brea, Calif.), INNOVN® and THROMBOREL® (Dade Behring, Liederbach, Germany). Methods of calculating INR are known to those skilled in the art. See, e.g., Cunningham, M T, Johnson, G F, Pennell, B J, and Olson, J D, Am J Clin Pathol., 102:128-33 (1994).

As used herein, the terms “activated partial thromboplastin time” or “aPTT” or “partial thromboplastin time” or “PTT,” refer to the time it takes the blood plasma of a subject to clot after the addition of a phospholipid (i.e., partial thromboplastin) and calcium chloride. Non-limiting examples of phospholipids for use in determining aPTT include silica and kaolin. See Eby, Charles, “Standardization of APTT Reagents for Heparin Therapy Monitoring: Urgent or Fading Priority?” Clinical Chem., 43(7):1105-1107 (1997).

INR grades are used to rank the severity thresholds for the coagulation factors relative to the upper normal of limit values (ULN). The higher the INR grade the higher the severity. In some embodiments, the INR in a subject is measured before and after the administration of a compound of formula (I) to the subject. In some embodiments, the INR in a subject is measured at 1 hour, 2 hour, 3 hour, 6 hour, 12 hour, 24 hour, or 48 hour intervals after administration of the compound of formula (I). In some embodiments, the INR in a subject after the administration of a compound of formula (I) to the subject is Grade 1, Grade 2, or Grade 3. As used herein, “Grade 1” refers to an INR value of 1.1 to 1.65 in a subject. As used herein, “Grade 2” refers to an INR value of 1.65 to 2.2 in a subject. As used herein, “Grade 3” refers to an INR value greater than 2.2 in a subject. In some embodiments, the INR in a subject after administration of a compound of formula (I) to the subject is a factor of an upper limit of normal value (“ULN”) in a subject. As used herein, “normal INR value” refers to the INR value in a subject that has not been administered a compound of formula (I). For example, if a subject has a normal INR value of 1 to 1.1, the upper limit of normal in the subject is 1.1. In some embodiments, the INR in a subject after the administration of a compound of formula (I) to the subject is 1.1 to 1.5 times, 1.5 to 2 times, or greater than 2 times the ULN in a subject.

In some embodiments, “Normal INR” refers to an INR of about 0.9 to about 1.1, or about 1 to about 1.1. In some embodiments, ULN is about 1.1.

In some embodiments, the use of therapeutic anticoagulants is aimed to achieve INR levels of between 2 and 3 (atrial fibrillation) or higher (valvular reconstruction). See, e.g., A. Garcia, et al., “The Risk of Hemorrhage Among Patients With Warfarin-Associated Coagulopathy” J American College of Cardiology 47:804 (2006).

In some embodiments, a method of the present invention is also directed to eliciting a dose response in a subject, the method comprising administering to the subject a compound of formula (I) as described above. A “dose response” refers to a direct or indirect correlation between a total daily dosage of compound of formula (I) administered to a subject and a desired clinical result in a subject. For example, in some embodiments, a dose response is defined as an ascending relationship between a total daily dosage of a compound of formula (I) administered to a subject and an INR value in the subject at a fixed interval of time. In some embodiments, if plotted on a graph, an ascending relationship would produce a plot in which the INR value in a subject (y-axis variable) versus the total daily dosage of a compound of formula (I) (x-axis variable) would display a positively sloped line or curve.

Without being bound to any particular theory, in some embodiments, continuous and consistent administration of the compound described herein can result in a decreasing anticoagulation effect (e.g., reduced INR values) over time, e.g., after 10 days. For example, in some embodiments, upon initial consistent and continuous administration of the compound of formula (I), the INR of a subject remains consistent (or increases for a time, e.g., 10 days, followed by a decrease in the INR value in the subject. For example, in some embodiments, the subject is administered a consistent amount of 0.25 g to 1 g of the compound of formula (I) at least twice a day, for 14 days, wherein the INR value in the subject remains constant (i.e., does not change by greater than 20%) during days 1 through 6 of the administration, increases during days 7 through 10 of the administration, and decreases during days 11 through 14 of the administration. Thus, in some embodiments, the anticoagulation effects of the compound of formula (I) are self limiting, since the anticoagulation effects decrease over time, and there is not the possibility of over dosing as long as the administration is continuous and consistent. In some embodiments, the INR profile of a subject being administered does not increase unexpectedly, and thus constant monitoring is not needed. In some embodiments, due to the decreasing anticoagulation effects of the compound of formula (I) over time, administration of the compound can continue, without interruption, while gradually decreasing the anticoagulation effect of the compound.

In some embodiments, the anticoagulation effects of the compound of formula (I) can be reversed by administration of a second active agent, e.g., administration of vitamin K, or a vitamin K analog.

The present invention is also directed to kits, or “therapeutic packages,” comprising greater than seven dosage forms, each dosage form comprising 0.25 g to 1 g of a compound of formula (I) as described above, and a label comprising directions for administering a compound of formula (I) to a subject according to the methods of the present invention. In some embodiments, the therapeutic package comprises greater than 14 dosage forms. In some embodiments, the therapeutic package comprises greater than 21 dosage forms. In some embodiments, the therapeutic package comprises greater than 30 dosage forms. In some embodiments, the therapeutic package comprises 7 to 93 dosage forms. In some embodiments, the therapeutic package comprises 14 to 62 dosage forms. In some embodiments, each dosage form comprises 0.25 g to 1 g of a compound of formula (I) and a label comprising directions for the use of the package for administering the compound to a subject according to the methods of the present invention. In some embodiments, the dosage forms are arranged for ease of use with daily administration, e.g., each day is clearly marked with 2, 3, or 4 dosage forms in each day. In some embodiments, a therapeutic package can comprise a cardboard or paper package with printed instructions. In some embodiments, a kit or therapeutic package can contain dosage forms, each dosage form of a constant amount of the compound of formula (I), or alternatively, different amounts of the compound of formula (I).

A “label” or “printed instructions” can be in a form prescribed by a governmental agency regulating the manufacture, use or sale of pharmaceuticals or biological products, which notice reflects approval by the agency of the manufacture, use or sale for human administration to reduce a symptom. The kit can further comprise printed matter, which, e.g., provides information on the use of a compound of formula (I), or a pre-recorded media device which, e.g., provides information on the use of a compound of formula (I).

“Printed matter” can be, for example, one of a book, booklet, brochure or leaflet. The printed matter can describe the use of a compound of formula (I) of the present invention to provide an anticoagulation effect in a subject. Possible formats included, but are not limited to, a bullet point list, a list of frequently asked questions (FAQ) or a chart. Additionally, the information to be imparted can be illustrated in non-textual terms using pictures, graphics or other symbols.

The present invention is further illustrated by the following Examples. These Examples are provided to aid in the understanding of the invention and are not to be construed as a limitation thereof.

### EXAMPLES

### Example 1

### Safety and Tolerability of Administration of (R,R,R) α-Tocopherol Quinone

The safety and tolerability of (R,R,R) α-tocopherol quinone was tested in healthy male and female subjects. Twelve subjects were divided into two groups of six subjects each (Groups A and B). Group A was administered 1.5 g of a (R,R,R) α-tocopherol quinone twice a day for one day. Group B was administered 1.0 g of (R,R,R) α-tocopherol quinone three times a day for 1 day. Both groups of subjects were fed under high and moderate fat meal content. Both Groups A and B tolerated the administration of (R,R,R) α-tocopherol quinone and no side effects were observed.

### Example 2

### Pharmacokinetics of TID Administration of a Total Daily Dosage of 1 g or 2 g of (R,R,R) α-Tocopherol Quinone

Subjects were divided into two groups of 6-10 subjects each (Groups C and D).

The administration schedule of (R,R,R) α-tocopherol quinone for each group is found in Table 1 below.

TABLE 1

| | Group C | Group D |
|---|---|---|
| Dosage Amount (g) | 0.33 | 0.67 |
| Dosage Frequency (per day) | 3 | 3 |
| Total Daily Dosage (g) | 1 | 2 |
| Length of Administration (days) | 7 | 7 |

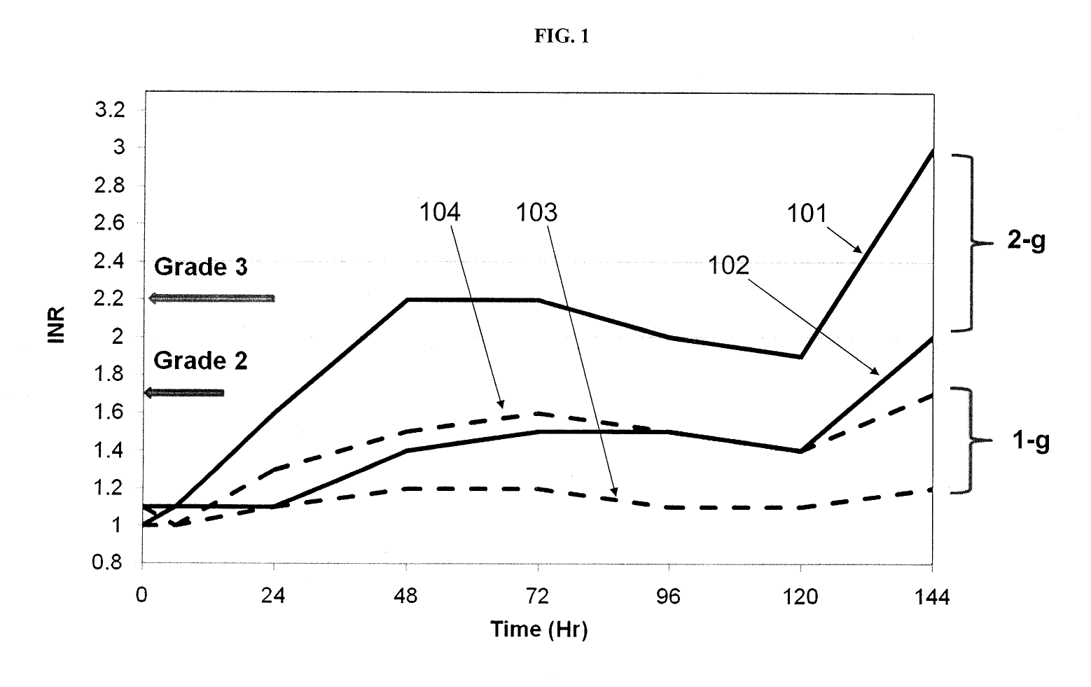

INR values were measured in the subjects at 24 hour intervals during the course of the administration. FIG. 1 represents a graphical description of the INR values for the subjects of Group C (dashed lines) and the subjects of Group D (solid lines). Solid line 101 represents the subject with the highest measured INR value. Solid line 102 represents the subject with the lowest measured INR value. Dashed line 103 represents the subject with the highest measured INR value. Dashed line 104 represents the subject with the lowest measured INR value.

### Example 3

### Pharmacokinetics of BID Administration of a Total Daily Dosage of 1 g or 1.5 g of (R,R,R) α-Tocopherol Quinone

Subjects were divided into two groups of 6 subjects each (Groups E and F). The administration schedule of (R,R,R) α-tocopherol quinone for each group is found in Table 2 below.

TABLE 2

| | Group E | Group F |
|---|---|---|
| Dosage Amount (g) | 0.5 | 0.75 |
| Dosage Frequency (per day) | 2 | 2 |
| Total Daily Dosage (g) | 1 | 1.5 |
| Length of Administration (days) | 14 | 14 |

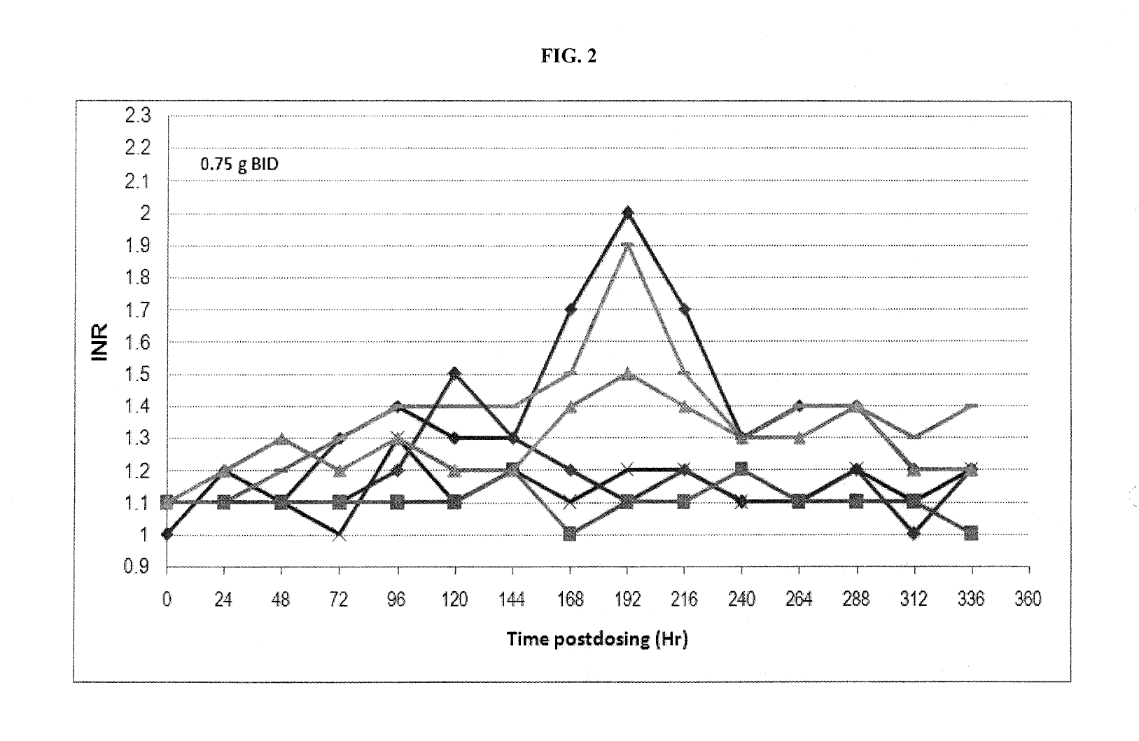

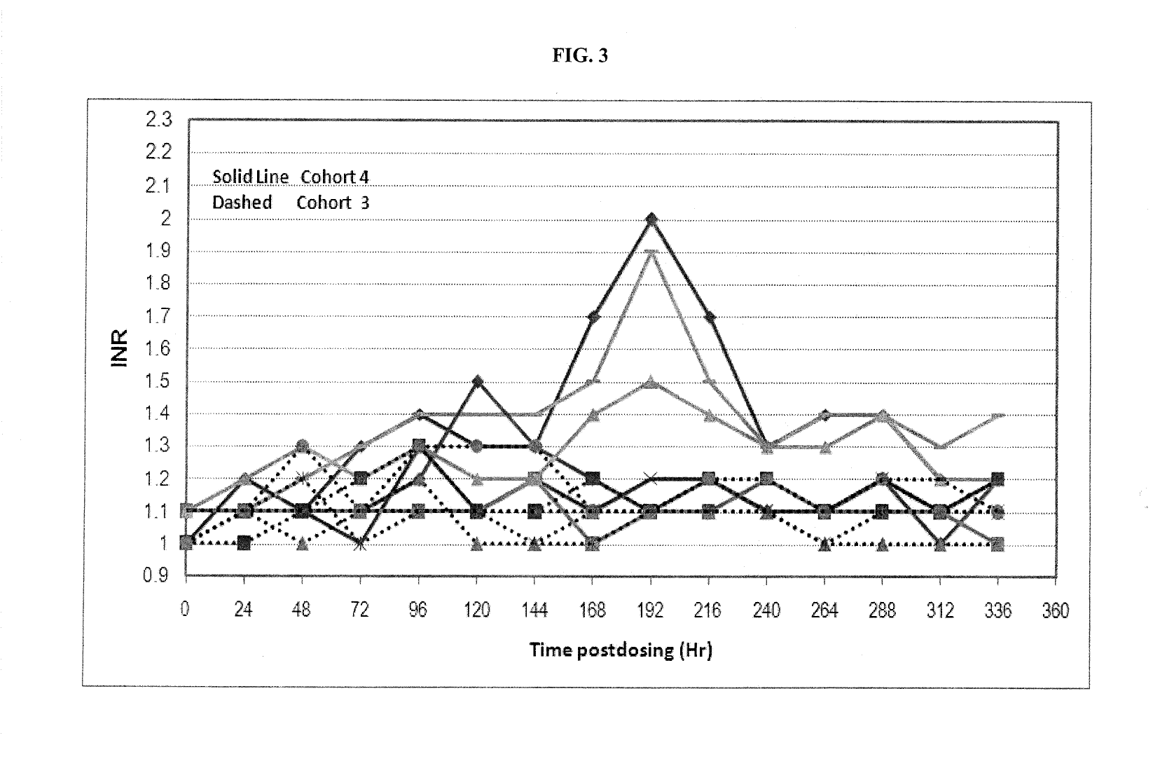

R values were measured in the subjects at 24 hour intervals during the course of the administration. FIG. 2 represents a graphical description of INR values for the 6 subjects of Group F. FIG. 3 represents a graphical description of the INR values for the subjects of Group E (dashed lines) and the subjects of Group F (solid lines).

### Example 4

### A Single-Blind, Randomized, Placebo-Controlled, Single-Dose, Ascending-Dose Study of the Safety, Tolerability, and Pharmacokinetic Effects in Healthy Volunteers

Healthy adult male subjects were enrolled into 1 of 6 cohorts of 10 subjects, and randomly assigned to receive either alpha-tocopherolquinone (ATQ) (8 subjects) in olive oil or placebo (2 subjects). Safety, tolerability and PK characteristics of ATQ were assessed under fasted conditions (low dose of 0.25 g and 0.5 g) and fed conditions (range of 0.5 g to 6.0 g), during co-administration with 400 IU vitamin E (2.0 g), and following a 2.2 g total dose administered in 3 equally-divided doses of 0.735 g, and 7 hours apart. There was a 2-week interval between dosing of cohorts while a medical review of the data collected on the preceding dose was conducted. Blood samples for the measurement of ATQ concentration were collected pre-dose and up to 168 hours following dose administration. All plasma ATQ concentrations were determined using a GLP validated bioanalytical method. Blood samples for the measurement of vitamin E concentrations were collected prior to and at 24 hours following dose administration (Cohorts 1, 2, and 3) and serially for up to 24 hours in Cohorts 4 through 6 except for Cohort 4, Period 2 (vitamin E co-administration) where samples were collected for up to 96 hours. Urine was collected for the measurement of ATQ and its metabolites in pooled intervals up to 72 hours post-dose for all cohorts.

Single oral ATQ doses (0.25 g and 0.5 g) were poorly absorbed under fasted conditions with maximum plasma concentrations ranging close to the background concentrations of endogenous α-tocopherolquinone. However, administration of 0.5 g ATQ with a high-fat meal increased bioavailability by over 60-fold. For this reason, it was decided to administer ATQ with a high-fat meal with all subsequent doses.

Following single ATQ doses of 0.5 g to 6.0 g administered after a high-fat breakfast, ATQ was slowly absorbed with a median Tₘₐₓ of 6 hours which was independent of dose. Absorption was dose proportional between 0.5 g and 1 g but was blunted at higher doses indicating dose-limited absorption. Administration of a 2.2 g daily dose of ATQ given as 3 equally-divided doses of 0.735 g, administered 7 hours apart (2.2 g total dose) rather than as a single 2.2 g unit, significantly increased ATQ bioavailability; in fact, the highest overall exposure (AUC) in this study was observed following the 2.2 g divided dose which slightly exceeded that following the single 6 g dose.

The dose-normalized exposure for the 2.2 g ATQ divided dose was comparable to dose levels of less than 1 g ATQ (i.e., 0.5 g ATQ administered under fed conditions) and clearly higher than seen following the 2 g and 6 g single doses.

The highest dose-normalized exposure (both Cₘₐₓ and AUC) and lowest CL/F was observed following the 1 g ATQ dose. Since dose-normalized exposure following the 2 g unit dose was lower than that of the 0.5 g unit dose and that of the 2.2 g divided dose (3×0.735 g), it can be assumed that the nonlinearity in absorption starts to occur at ATQ unit doses of greater than 1 g.

Following ATQ administration under fed conditions, mean t_{1/2} values ranged from 12.7 to 36.2 hours. ATQ t_{1/2} was independent of dose. The long apparent terminal t_{1/2} values calculated in this study do not significantly contribute to ATQ exposure. ATQ concentrations essentially returned to endogenous α-tocopherolquinone baseline levels within 24 hours post-dose and baseline-adjusted ATQ concentrations at 24 hours tended to account for less than 1% of Cₘₐₓ indicating that ATQ has a shorter effective half-life than that described by the terminal elimination profile. Mean ATQ pharmacokinetic parameters under fed conditions are summarized in Table 3. Unchanged ATQ was not detected in any of the urine samples analyzed in this study.

TABLE 3
Mean (% CV) ATQ Pharmacokinetic
Parameters under Fed Conditions

| | | | | | 2.2 g |
|---|---|---|---|---|---|
| Parameter | | | | | (0.735 g |
| (N = 8) | 0.5 g | 1.0 g | 2.0 g | 6.0 g | TID) |
| Cₘₐₓ | 4.549 | 13.14 | 13.83 | 18.65 | 10.99 |
| (μg/mL) | (42.5) | (34.3) | (40.0) | (16.0) | (49.2) |
| Tmax | 6.0 | 6.0 | 6.0 | 6.0 | 16.0 |
| (h)[a] | 4.0-6.0 | 4.0-8.0 | 4.0-12.0 | 4.0-6.0 | 3.0-20.0 |
| AUC₍₀₋ₗₐₛₜ₎ | 22.99 | 64.65 | 69.6 | 100.0 | 106.8 |
| (μg*h/mL) | (39.4) | (25.9) | (49.7) | (22.7) | (33.0) |
| AUC_{(0-inf)} | 22.43[b] | 66.70[c] | 72.3[c] | 101.3[b] | 107.7 |
| (μg*h/mL) | (46.8) | (26.1) | (50.8) | (28.0) | (33.1) |
| t_{1/2} (h) | 27.692[b] | 36.243[c] | 25.633[c] | 29.259[b] | 29.951 |
| | (56.2) | (38.0) | (44.9) | (58.2) | (15.7) |
| CL/F | 25.910[b] | 15.950[c] | 33.404[c] | 62.824[b] | 22.731 |
| (L/h) | (37.9) | (27.4) | (41.2) | (24.7) | (35.8) |

[a]mean and range reported;
[b]N = 6;
[c]N = 7

The ATQ concentration versus time profile was essentially unchanged when ATQ (2 g QD dose) was administered with and without 400-IU of vitamin E indicating the lack of any clinically relevant effect of vitamin E on ATQ pharmacokinetics. Likewise no effect was noted on endogenous vitamin E concentrations following administration of ATQ across all dose groups. Furthermore, there was no obvious difference in vitamin E exposure between the 8 subjects receiving 2 g ATQ and 400-IU of vitamin E and the 2 subjects receiving 400-IU of vitamin E and placebo.

### ATQ Single Dose and INR

ATQ elicited a slight but not clinically significant effect on the coagulation tests, especially PT and INR. Of the 48 subjects who received ATQ, 11 subjects had postdose PT values above the upper limit of normal (ULN), 2 subjects had a 24-hour postdose INR value above ULN, and 1 subject had a 24-hour postdose PTT value above ULN.

In Cohort 3 (1-g ATQ), 2 of 8 subjects had a 24-hour postdose PT value which was 5.2% above the upper limit of normal (ULN).

In Cohort 5 (6-g ATQ), 5 of 8 subjects had postdose PT values which were above ULN. Two of the subjects had either a screening or Day −1 PT value which was above ULN. The subject with the highest postdose PT value (39.1% above ULN) also had a corresponding increase in INR (23.1% above ULN) and PTT (5.6% above ULN), respectively, all of which were considered clinically relevant as described above.

In Cohort 6 (2.2-g as 0.735-g TID), 4 of 8 subjects had postdose PT values which were above ULN. The subject with the highest postdose PT value (20.9% above ULN) also had a corresponding increase in INR (7.7% above ULN). NO INR change was observed when 2 g was administered as a single entity (2 g QD)

The INR, and PTT values returned to normal range by 48-hour postdose in all subjects; the PT values returned to normal range by 48-hour postdose in the majority of subjects. All changes in hemostasis were fully reversible upon cessation of the treatment. No formal Maximal Tolerated Dose level was established.

### Example 5

### A Single-Blind, Randomized, Placebo-Controlled Ascending Oral Multiple-Dose and Food-Effect Study Assessing the Safety, Tolerability, and Pharmacokinetics of ATQ in Healthy Male and Female Subjects

A study was conducted with the goal to assess safety, tolerability, and PK of multiple-ascending oral doses of ATQ as well as to evaluate the pharmacodynamic effects of ATQ on hemostasis and to determine the maximum tolerated daily dose. The effect of ATQ on endogenous Vitamin E levels during extended ATQ dose administration was also assessed.

The study was conducted in 2 parts. Part 1 was conducted in healthy male subjects only while Part 2 was conducted in healthy male and female subjects. The intent of this study part was to optimize the dosage regimen and diet for the multiple-dose safety and tolerance Part 2 of the study. All plasma ATQ concentrations were determined using a GLP validated bioanalytical method. ATQ was administered as an oral solution in oil (0.49 g/mL). The placebo solution consisted of olive oil alone.

### Part 1: Food Effect

Part 1 of the study was a randomized, open-label, 2-sequence, 2-way complete crossover design in 12 healthy male subjects to compare the relative bioavailability of a single 3-g ATQ daily dose when administered with either a standard or high-fat meal as a twice-daily dose (BID) (2×1.5-g 12 hours apart) or 3 times daily (TID) (3×1.0-g 6 hours apart). In Example 4, a significant food effect had been demonstrated and the intent of this study part was to optimize the dosage regimen and diet for the multiple-dose safety and tolerance Part 2 of the study. The dose of 3-g ATQ was selected for Part 1 as it most likely represented the high end of the exposure spectrum selected for the subsequent tolerance trial. For Part 1, serial blood samples for the measurement of the plasma levels of ATQ were collected prior to and following each oral dose administered on Day 1 through 36 hours following the morning dose on Day 1 (or 24 hours post-evening dose).

Maximum exposure (mean Cₘₐₓ range 13.4 to 15.3 μg/mL) occurred at a median Tₘₐₓ of 6.00 to 17.50 hours, but varied greatly across subjects and treatments. ATQ was rapidly eliminated as shown by the short mean t_{1/2} values of 4.20 to 5.37 hours. Apparent oral clearance and Vz/F were similar across treatments.

There was no difference in ATQ exposure (Cₘₐₓ and AUC) when administered following either a high-fat or standard meal. Furthermore, overall exposure (AUC) was similar when the 3-g dose was administered either as 2×1.5-g doses every 12 hours or 3×1.0-g doses every 6 hours. Key ATQ baseline-adjusted plasma pharmacokinetic parameters (arithmetic mean [% CV]) are provided in Table 4.

TABLE 4
Baseline-adjusted plasma pharmacokinetic parameters (arithmetic mean
[% CV]) obtained in Part 1 following a single 3-g daily ATQ dose

| Parameter | Group 1: | | Group 1: | | Group 2: | | Group 2: | |
|---|---|---|---|---|---|---|---|---|
| Mean (% CV) | 3-g BID-HF | | 3-g BID-SM | | 3-g TID-HF | | 3-g TID-SM | |
| (unit) | (N = 6) | | (N = 6) | | (N = 5) | | (N = 6) | |
| Cₘₐₓ (μg/mL) | 13.4 | (33.8) | 15.3 | (34.3) | 14.2 | (32.9) | 15.0 | (17.1) |
| Tₘₐₓ (h)[a] | 10.50 | (3.00-21.00) | 6.00 | (4.50-15.00) | 17.50 | (5.95-21.00) | 12.73 | (5.95-19.00) |
| AUC_{(0-∞)} | 152 | (33.2) | 157 | (45.9) | 178 | (30.8) | 169 | (15.0) |
| (μg · h/mL) | | | | | | | | |
| t_{1/2} (h) | 4.20 | (35.9) | 4.34 | (27.7) | 4.88 | (24.8) | 5.37 | (54.8) |
| CLₚₒ (L/h) | 21.4 | (29.2) | 22.0 | (38.0) | 18.2 | (30.4) | 18.1 | (14.9) |
| Vz/F (L) | 132 | (50.7) | 143 | (52.1) | 122 | (18.8) | 139 | (57.0) |

Notes:
BID = 2 equally-divided doses administered 12 hours apart or TID = 3 equally-divided doses administered 6 hours apart; HF = high-fat meal; SM = standard meal
[a]Median (range).

### Part 2: Multiple Ascending ATQ Dose

Part 2 was conducted as a multiple-dose dose-escalation design. Based on the Part 1 results, Part 2 proceeded with a TID regimen administered following a standard breakfast, lunch, and dinner dosed through the morning of Day 7. Dose administration was subsequently switched to a BID regimen administered for 14 days.

Part 2 was conducted as a multiple-dose dose-escalation design in male and female subjects. None of the subjects who participated in Part 1 of the study were eligible to participate in Part 2. A total of 32 subjects were enrolled in Part 2. Twenty (20) subjects received ATQ or placebo for one week in a single-blind, randomized, placebo-controlled design: Cohort 1 (N=8) low dose (1 g/day as 0.33 g TID) and Cohort 2 (N=8) midrange dose (2 g/day as 0.67 g TID). In each cohort, 8 subjects received ATQ and 2 subjects received placebo under fed conditions. An additional 12 subjects were subsequently enrolled into 2 additional cohorts of 6 subjects each. The 12 subjects were randomized to receive one of the two following ATQ doses: Cohort 3 (N=6) low dose (0.5 g BID) and Cohort 4 (N=6) intermediate dose (0.75 g BID) administered for 14 days with a standard meal. All subjects participating in Cohorts 3 and 4 received ATQ; there was no placebo arm.

For Part 2, serial blood samples for the measurement of the plasma levels of ATQ were collected in Cohorts 1 and 2 as part of a 3 times daily regimen prior to and over the first 6-hour dosing interval following administration of the first dose on Day 1, prior to the morning dose on Days 3, 5, and 6, over the 6-hour and 12-hour dosing intervals following the afternoon and evening dose, respectively, and up to 48 hours after the final (morning) dose on Day 7.

In Cohorts 3 and 4, serial blood samples for the measurement of the plasma levels of ATQ were collected for the twice-daily regimen prior to and over the 12-hour dosing interval following the first dose administration on Day 1, prior to the morning dose on Days 4, 7, 10, 13, and 14, and over the 12-hour dosing interval after the final (morning) dose on Day 14. Multiple-dose PK data over the dose range of 1.0-g/day (BID and TID), 1.5-g/day (BID), and 2.0-g/day (TID) were generated in this study.

Relevant arithmetic mean (% CV) multiple-dose ATQ pharmacokinetic parameters are provided in Table 5 below. Part 2 multiple-dose results are provided for Day 7 (Cohorts 1 and 2) or Day 14 unless otherwise indicated. Parameters designated with the term 24 refer to pharmacokinetic parameters derived for a 24-hour TID or BID dosing period.

TABLE 5
Arithmetic mean (% CV) multiple-dose ATQ pharmacokinetic parameters

| Part 2—Multiple Dose | 1-g/day | | 2-g/day | | 1-g/day | | 1.5-g/day | |
|---|---|---|---|---|---|---|---|---|
| Parameter in Part 2 | (0.33 g TID) | | (0.67 g TID) | | (0.5 g BID) | | (0.75 g BID) | |
| (unit) | (N = 8) | | (N = 7) | | (N = 5) | | (N = 6) | |
| Cₘₐₓ (μg/mL) [a] | 3.53 | (38.7) | 9.00 | (56.6) | 1.89 | (63.1) [f] | 2.54 | (51.5) [f] |
| | (Day 6 lunch) | | (Day 6 dinner) | | | | | |
| Cₘₐₓ last dose (μg/mL) | 2.40 | (44.8) | 4.28 | (51.8) | 1.89 | (63.1) | 2.54 | (51.5) |
| TimeHgh₍₀₋₂₄₎ (h) [b] | 23.80 | (17.98-24.24) | 23.87 | (20.08-23.92) | 19.68 [e] | | 21.84 [e] | |
| AUC_{(0-tau, 24)} (μg · h/mL) | 41.4 | (37.1) | 108 | (43.3) | 25.0 [e] | | 33.2 [e] | |
| CL_{po, 24} (L/h) | 24.2 [c] | | 18.5 [c] | | 57.5 | (64.0) [f] | 53.5 | (45.6) [f] |
| C_{avg, 24 h} (μg/mL) | 1.73 | (37.1) | 4.48 | (43.2) | 1.04 | (63.7) [f] | 1.38 | (45.6) [f] |
| Fluct_{24 h} (%) | 189 | (31.2) | 177 | (28.2) | 145 | (16.0) [f] | 147 | (19.0) [f] |
| Cₘᵢₙ (μg/mL) | 0.583 | (67.9) | 1.22 | (42.1) | 0.308 | (45.7) | 0.443 | (37.8) |

Notes:
BID = twice daily; NA = not applicable; TID = 3 times daily; TimeHgh = time above the pharmacologically relevant concentration of 0.5 μg/mL
[a] Highest Cₘₐₓ value of the 3 serial TID profiles; associated with lunch or dinner as indicated above.
[b] Median (range).
[c] Calculated as the daily dose divided by mean AUC_{(0-tau, 24)} for the TID regimen.
[d] Ratio (90% CI): comparison made for the Day 7 or Day 14 AM dose versus the Day 1 AM dose. Accumulation was assessed using single repeated measures linear mixed effects model for each parameter with day as fixed-repeated effect. Values represent the point estimates of the geometric LS mean ratios.
[e] Calculated as 2 times the value obtained following the 0-12 hour BID morning dose (assumed that the morning dose BID results are reflective of those following the evening dose).
[f] Value represents the results of the morning dose pharmacokinetic data and is assumed to be representative for the 24-hour dosing interval.

ATQ elicited a slight but not clinically significant effect on the coagulation tests, especially PT and INR. Of the 48 subjects who received ATQ, 11 subjects had postdose PT values above the upper limit of normal (ULN), 2 subjects had a 24-hour postdose INR value above ULN, and 1 subject had a 24-hour postdose PTT value above ULN.

In Cohort 3 (1-g ATQ), 2 of 8 subjects had a 24-hour postdose PT value which was 5.2% above the upper limit of normal (ULN).

In Cohort 5 (6-g ATQ), 5 of 8 subjects had postdose PT values which were above ULN. Two of the subjects had either a screening or Day −1 PT value which was above ULN. The subject with the highest postdose PT value (39.1% above ULN) also had a corresponding increase in INR (23.1% above ULN) and PTT (5.6% above ULN), respectively, all of which were considered clinically relevant as described above.

In Cohort 6 (2.2-g as 0.735-g TID), 4 of 8 subjects had postdose PT values which were above ULN. The subject with the highest postdose PT value (20.9% above ULN) also had a corresponding increase in TNR (7.7% above ULN).

The INR, and PTT values returned to normal range by 48-hour postdose in all subjects; the PT values returned to normal range by 48-hour postdose in the majority of subjects. All changes in hemostasis were fully reversible upon cessation of the treatment. No formal Maximal Tolerated Dose level was established.

Following multiple dose administration (Part 2), ATQ trough concentrations appeared to reach steady state within 3 days for the 0.33 g TED and 0.67 g TID dose groups, 4 days for the 0.5 g BID dose group but not until Day 13 for the0.75 g BID, dose group. Mean trough concentrations observed in the TID regimen were higher than those in the BID regimen at the same daily ATQ dose.

Within the respective dosing regimen (TID or BID), the increases over the 2-fold (TID) or 1.5-fold (BID) dose range resulted in approximately proportional increases in the ATQ exposure following multiple-dose administration. Across the different dosing frequencies, however, exposure following TID administration was higher at similar doses. Maximum concentrations were approximately 27% higher following the final morning dose for both regimens. The highest mean Cₘₐₓ value in the TID regimen occurred after lunch and was close to twice that observed in the BID regimen. The estimated daily AUC was approximately 66% higher following the 1-g/day for the 0.33 g TID than the 0.5 g BID regimen.

The accumulation ratio decreased with increasing daily TID dose and was lower for the BID regimen at the same daily doses. This is most likely due to the incomplete characterization of the Day 1 6-hour pharmacokinetic profile. Following BID administration, Cₘₐₓ did not accumulate and accumulation on AUC was approximately 43% to 66%.

### Pharmacodynamic Effects of ATQ on Hemostasis:

Based on the results of the Example 1 and data from the 4 week rat and dog studies, ATQ appeared to have an effect on coagulation. Thus both the basic coagulation panel assessments including PT, INR, aPTT, and platelet counts were performed at screening, on Day −1, and extensively throughout the treatment period to assess the potential effect of ATQ on coagulation. In addition, an extended coagulation biomarker panel assessing various factors of coagulation as well as overall platelet function was included in the study as a means to understand the underlying mechanism of the observed INR and PT increases in the clinic.

ATQ associated changes in coagulation as pertaining to the safety and tolerance of ATQ are discussed below. This section covers the PD effect of ATQ on the coagulation parameters PT, INR, and aPPT and the relative time course of drug induced changes in these parameters.

The following exploratory coagulation biomarkers (extended coagulation panel) were included in the clinical laboratory tests to elucidate the potential effects of ATQ on coagulation factors I (fibrinogen), II (prothrombin), V, VII, IX, and X, protein C, protein S, antithrombin (AT), and platelet function (PF).

In Part 1 the extended panel was collected at screening and at check-in (Day −1) and Day 2 of each period unless the study results warranted a different sampling schedule. In Part 2, the same of the exploratory coagulation biomarkers were collected at screening, check-in, Day 4, and Day 8 in Cohorts 1 and 2, or at check-in, Day 7, and Day 14 in Cohorts 3 and 4 with the following exception: in Cohorts 3 and 4, platelet function was collected at check-in only or when medically indicated.

ATQ administered either as 3-g over 1 day, 0.33 g TID and 0.67 g TID (1- to 2-g/day) or 0.75 g BID (1.5-g/day) exerted a clear effect on coagulation. The effect was manifested by increases in INR, PT, and, to a lesser extent, aPTT. The most pronounced changes were seen in the 0.67 g TID group with 3 of 8 subjects reaching INR values of 2.3, 3.0 and 3.0, respectively. These levels were more than two times the normal level of INR and per protocol the subjects were discontinued.

Onset of the effect of ATQ on coagulation occurred as early as 18 hours following the first (high dose) of ATQ (Part 1) or by Day 2 of multiple dose administration. The effect of ATQ on coagulation appeared to be biphasic with an initial increase in INR, PT, and aPTT through Day 4, followed by a second peak by Day 7 to 8 of dose administration. When ATQ was administered for 14 days at the dose levels of 0.5 g and 0.75 g BID, the effect on coagulation appeared to slowly abate while still on drug beyond 8 days of dose administration. Upon drug cessation, effects on coagulation (INR, PT, and aPTT) showed a prompt downwards trend with most subjects approaching baseline coagulation values at 48 hours post last dose.

When looking in more detail at the coagulation factors, it appeared that the changes were restricted to decreases in the activity of the Vitamin K-dependent coagulation factors II, VII, IX, X and related substances, protein C and protein S. In Part 1 (3-g over one single day), decreases in activity (across BID and TID treatments) were as follows: protein C (70 to 97%), factor VII (55 to 74%), factor IX (41 to 59%), protein S (47 to 50%), factor X (28 to 38%). The effect of ATQ on the activity of factor II was small (10 to 20% decrease). In Part 2, multiple-dose administration of 1 to 2-g/day ATQ also revealed a dose dependent effect on the Vitamin K dependent coagulation factors and related substances. The greatest decreases from baseline activity were observed following administration of 0.67 g TID while on drug (Day 4): protein C (109%), factor IX (79%), factor VII (75%), factor X (62%; 68% on Day 14), protein S (56%), and factor II (51%). The fact that some factors show decreases earlier than others is entirely consistent with their half lives, with Protein C and factor VII having the shortest half life, and factor II the longest.

Administration of ATQ did not appear to affect fibrinogen (factor I) concentrations or the activity of factor V or AT.

### Coagulation and Safety:

Increases in coagulation parameters International Normalized Ratio and prothrombin time were noted in most subjects following dosing across treatments. Twelve subjects receiving ATQ during Part 1 of the study had clinically relevant changes in clinical laboratory values related to treatment-emergent adverse events that were assessed as possibly or probably related to study drug. Of these, 5 subjects had clinical coagulation laboratory values greater than 1.5 times the upper limit of normal (value greater than 1.65).

Twenty-five active-treated subjects had clinically relevant changes in clinical laboratory values related to treatment-emergent adverse events that were assessed as possibly or probably related to study drug. Of these, 11 subjects receiving ATQ had study-drug related clinical coagulation laboratory values greater than 1.5 times the upper limit of normal (value greater than 1.65).

Single daily doses of 3-g ATQ were associated with a Grade 2 increase in INR in 4 of 12 subjects. Following multiple daily administration of 2-g/day (0.67 g TID), increases in INR reached an intensity of Grade 3 in 3 of 8 active-treated subjects which resulted in discontinuation of further dose escalation. Changes in coagulation were less pronounced following 0.75 g BID administration of ATQ where the maximum increase in INR reached an intensity of Grade 2 in 2 of 6 subjects, as assessed by the Investigator. This effect was transient since INR values tended to decrease beyond 8 days of drug administration. Upon drug cessation, these changes showed a prompt downwards trend with most subjects approaching baseline coagulation values at 48 hours.

All of the various embodiments or options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A method of providing an anticoagulation effect in a subject in need thereof, comprising administering to the subject at least twice a day a compound of formula (I): 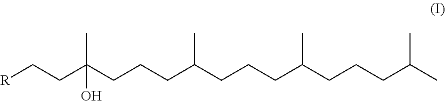
wherein R is selected from: 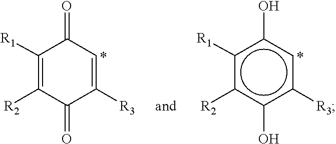
wherein the * indicates the point of attachment of R; and
R₁, R₂, and R₃ are independently selected from H, C₁-C₆ alkyl, and C₁-C₆ haloalkyl.

2. The method of claim 1, wherein if any one of R₁, R₂, and R₃ is H, then at least one other of R₁, R₂, and R₃ is neither H nor methyl.

3. The method of any one of claims 1 and 2, wherein the compound is administered to the subject at least three times a day.

4. The method of any one of claims 1 to 3, wherein a total daily dosage of 0.2 g to 12 g of the compound is administered to the subject.

5. The method of claim 4, wherein 0.1 g to 6 g of the compound is administered to the subject at least twice a day.

6. The method of claim 5, wherein 0.5 g to 4 g of the compound is administered to the subject at least twice a day.

7. The method of claim 4, wherein 0.1 g to 4 g of the compound is administered to the subject three times a day.

8. The method of claim 7, wherein 0.3 g to 2 g of the compound is administered to the subject three times a day.

9. The method of any one of claims 1 to 8, wherein R is: 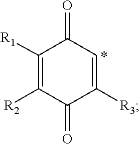
and
R₁, R₂, and R₃ are independently selected from H and C₁-C₂ alkyl.

10. The method of claim 9, wherein the compound of formula (I) is:

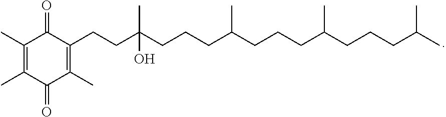

11. The method of claim 10, wherein the compound of formula (I) is:

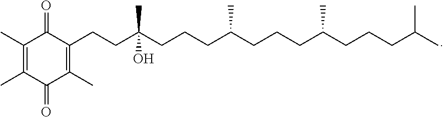

12. A method of treating thrombosis in a subject in need thereof, comprising administering to the subject at least twice a day a compound of formula (I): 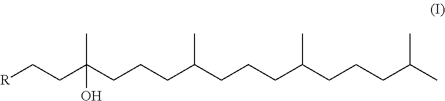
wherein R is selected from: 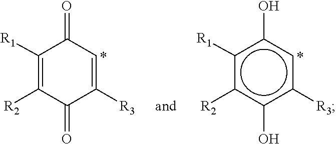
wherein the * indicates the point of attachment of R; and
R₁, R₂, and R₃ are independently selected from H, C₁-C₆ alkyl, and C₁-C₆ haloalkyl.

13. The method of claim 12, wherein if any one of R₁, R₂, and R₃ is H, then at least one other of R₁, R₂, and R₃ is neither H nor methyl.

14. The method of any one of claims 12 and 13, wherein the thrombosis is selected from the group consisting of venous thrombosis, deep vein thrombosis, renal vein thrombosis, arterial thrombosis, and combinations thereof.

15. The method of any one of claims 12 to 14, wherein the compound is administered to the subject at least three times a day.

16. The method of any one of claims 12 to 15, wherein a total daily dosage of 0.2 g to 12 g of the compound is administered to the subject.

17. The method of claim 16, wherein 0.1 g to 6 g of the compound is administered to the subject at least twice a day.

18. The method of claim 17, wherein 0.5 g to 4 g of the compound is administered to the subject at least twice a day.

19. The method of claim 16, wherein 0.1 g to 4 g of the compound is administered to the subject three times a day.

20. The method of claim 19, wherein 0.3 g to 2 g of the compound is administered to the subject three times a day.

21. A method of treating thrombosis in a subject in need thereof, comprising administering to the subject at least twice a day 0.1 g to 6 g of a compound of formula (I), wherein the compound is:

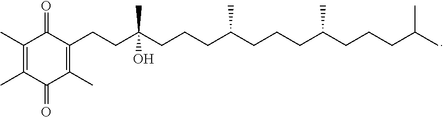

22. A method of treating a subject at risk of a condition selected from the group consisting of stroke, myocardial infarction, complications associated with cardiac valve replacement, and combinations thereof, the method comprising administering to a subject at least twice a day a compound of formula (I): 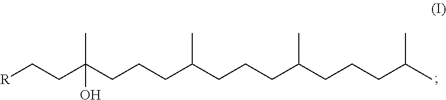
wherein R is selected from: 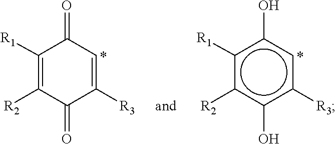
wherein the * indicates the point of attachment of R; and
R₁, R₂, and R₃ are independently selected from H, C₁-C₆ alkyl, and C₁-C₆ haloalkyl.

23. The method of claim 22, wherein if any one of R₁, R₂, and R₃ is H, then at least one other of R₁, R₂, and R₃ is neither H nor methyl.

24. The method of any one of claims 1 to 23, wherein the compound is administered to the subject orally, nasally, via inhalation, parenterally, subcutaneously, intramuscularly, transdermally, or buccally.

25. The method of any one of claims 1 to 24, wherein an oral dosage form comprising the compound of formula (I) is administered to the subject.

26. The method of any one of claims 1 to 25, wherein the method further comprises measuring in the subject at least one coagulation factor selected from the group consisting of Factor I, Factor II, Factor V, Factor VII, Factor IX, Factor X, Protein C, Protein S, antithrombin, platelet function, and combinations thereof.

27. The method of any one of claims 1 to 26, wherein the method further comprises measuring the international normalized ratio (INR), prothrombin time (PT), activated partial thromboplastin time (aPTT), and combinations thereof in the subject.

28. The method of any one of claims 1 to 27, wherein the compound of formula (I) is a stereoisomer thereof.

29. The method of any one of claims 1 to 28, wherein the compound of formula (I) is a racemate thereof.

30. The method of any one of claims 1 to 29, wherein the method comprises administering the compound formula (I) to the subject for a period of less than 20 days.

31. A therapeutic package comprising:
(a) greater than seven dosage forms, each dosage form comprising 0.1 g to 6 g of a compound of formula (I): 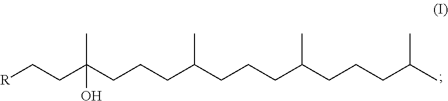
wherein R is selected from: 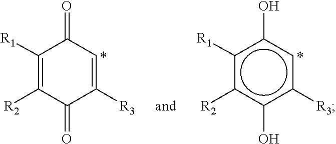
wherein the * indicates the point of attachment of R;
R₁, R₂, and R₃ are independently selected from H, C₁-C₆ alkyl, and C₁-C₆ haloalkyl; and
(b) a label comprising directions for administering the compound to a subject according to any one of the methods of claims 1-30.

32. The therapeutic package of claim 31, wherein if any one of R₁, R₂, and R₃ is H, then at least one other of R₁, R₂, and R₃ is neither H nor methyl.
